(19) United States
(12) Patent Application Publication
(10) Pub. No.: US 2009/0143421 A1
(43) Pub. Date: Jun. 4, 2009

(54) USE OF 2-BENZOYL-IMIDAZOPYRIDINES IN THERAPEUTICS

(75) Inventor: Jean-Francois PEYRONEL, Antony (FR)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: SANOFI-AVENTIS, Paris (FR)

(21) Appl. No.: 12/336,998

(22) Filed: Dec. 17, 2008

Related U.S. Application Data
(63) Continuation of application No. PCT/FR2007/001124, filed on Jul. 3, 2007.

(30) Foreign Application Priority Data
Jul. 3, 2006 (FR) 0606011

(51) Int. Cl. A61K 31/437; A61P 25/00; A61P 29/00; A61P 35/00; A61P 19/10

(52) U.S. Cl. 514/300

(57) ABSTRACT

The present invention is related to the use of a compound of formula (I):

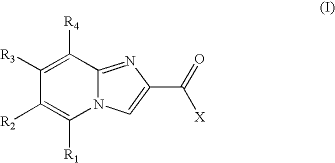

wherein R₁, R₂, R₃, R₄ and X are as defined herein, or an addition salt with an acid, for treating or preventing illnesses involving the nuclear receptors Nurr-1, also known as NR4A2, NOT, TINUR, RNR-1, and HZF3.

This application is a Continuation of International Application No. PCT/FR2007/001124, filed Jul. 3, 2007, which is incorporated herein by reference in its entirety.

### FIELD OF THE INVENTION

The present invention relates to the application in therapeutics of 2-benzoylimidazo[1,2-a]pyridine derivatives in the treatment or prevention of illnesses involving the nuclear receptors Nurr-1, also known as NR4A2, NOT, TINUR, RNR-1, and HZF3.

### SUMMARY OF THE INVENTION

The subject of the present invention is the use of compounds corresponding to the formula (I):

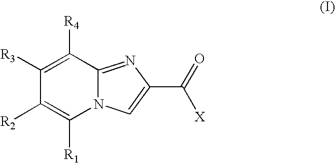

X is a phenyl, R₁, R₂, R₃ and R₄ are hydrogen; or
X is a phenyl, R₃ is methyl and R₁, R₂ and R₄ are hydrogen; or
X is a phenyl, R₂ is chlorine or methoxy and R₁, R₃ and R₄ are hydrogen; or
X is a phenyl, R₂ and R₃ are methoxy and R₁ and R₄ are hydrogen; or
X is a phenyl, R₁ is methoxy and R₂, R₃ and R₄ are hydrogens; or
X is a phenyl, R₃ is methoxy and R₁, R₂ and R₄ are hydrogen; or
X is a 4-methylphenyl, R₂ is a methyl and R₁, R₃ and R₄ are hydrogen; or
X is a 4-chlorophenyl, R₁ is a chlorine or a methoxy or a methyl, and R₂, R₃ and R₄ are hydrogen; or
X is a 4-chlorophenyl, R₂ is a chlorine or a methyl and R₁, R₃ and R₄ are hydrogen; or
X is a 4-chlorophenyl, R₃ is a methyl, and R₁, R₂ and R₃ are hydrogen; or
X is a 4-chlorophenyl, R₄ is a methyl and R₁, R₂ and R₄ are hydrogen; or
X is a 4-chlorophenyl, R₁ and R₃ are methyl and R₂ and R₄ are hydrogen; or
X is a 4-chlorophenyl and R₁, R₂, R₃ and R₄ are hydrogens; or
X is a 2-chlorophenyl and R₁, R₂, R₃ and R₄ are hydrogen; or
X is a 4-methylphenyl and R₁, R₂, R₃ and R₄ are hydrogens,
in the form of a base or of an addition salt with an acid for the preparation of a medication for treating and preventing diseases in which the NOT receptor is involved.

### DETAILED DESCRIPTION OF THE INVENTION

Among the compounds of formula (I) that are the subject of the invention, a first group of compounds is composed of compounds for which:
X is a phenyl, R₁, R₂, R₃ and R₄ are hydrogens; or
X is a phenyl, R₂ is chlorine or methoxy and R₁, R₃ and R₄ are hydrogens;
in the form of a base or of an addition salt with an acid.

The compounds of formula (I) may exist in the form of bases or of addition salts with acids. Such addition salts form part of the invention.

These salts may be prepared with pharmaceutically acceptable acids, but the salts of other acids useful, for example, for purifying or isolating the compounds of formula (I) also form part of the invention.

The compounds of formula (I) may also exist in the form of hydrates or solvates, namely in the form of associations or combinations with one or more water molecules or with a solvent. Such hydrates and solvates also form part of the invention.

Among the compounds of formula (I) that are the subject of the invention, mention may especially be made of the following compounds:
(5-methoxyimidazo[1,2-a]pyridin-2-yl)(phenyl)methanone;
(7-methoxyimidazo[1,2-a]pyridin-2-yl)(phenyl)methanone;
(6,7-dimethoxyimidazo[1,2-a]pyridin-2-yl)(phenyl)-methanone and its hydrobromide (1:1);
(imidazo[1,2-a]pyridin-2-yl)(phenyl)methanone and its hydrochloride (1:1);
(6-chloroimidazo[1,2-a]pyridin-2-yl)(phenyl)methanone and its hydrobromide (1:1);
(6-methoxyimidazo[1,2-a]pyridin-2-yl)(phenyl)methanone;
(7-methylimidazo[1,2-a]pyridin-2-yl)(phenyl)methanone;
(6-methylimidazo[1,2-a]pyridin-2-yl)(4-methylphenyl)-methanone;
(4-chlorophenyl)(6-methylimidazo[1,2-a]pyridin-2-yl)-methanone;
(6-chloroimidazo[1,2-a]pyridin-2-yl)(4-chlorophenyl)-methanone;
(5-chloroimidazo[1,2-a]pyridin-2-yl)(4-chlorophenyl)-methanone;
(4-chlorophenyl)(5-methoxyimidazo[1,2-a]pyridin-2-yl)-methanone;
(4-chlorophenyl)(5-methylimidazo[1,2-a]pyridin-2-yl)-methanone;
(4-chlorophenyl)(8-methylimidazo[1,2-a]pyridin-2-yl)-methanone;
(4-chlorophenyl)(7-methylimidazo[1,2-a]pyridin-2-yl)-methanone;
(4-chlorophenyl)(5,7-dimethylimidazo[1,2-a]pyridin-2-yl) methanone;
(imidazo[1,2-a]pyridin-2-yl)(4-methylphenyl)methanone; (4-chlorophenyl)(imidazo[1,2-a]pyridin-2-yl)methanone; and
(2-chlorophenyl)(imidazo[1,2-a]pyridin-2-yl)methanone.

In accordance with the invention, it is possible to prepare the compounds of general formula (I) according to the process described in scheme 1.

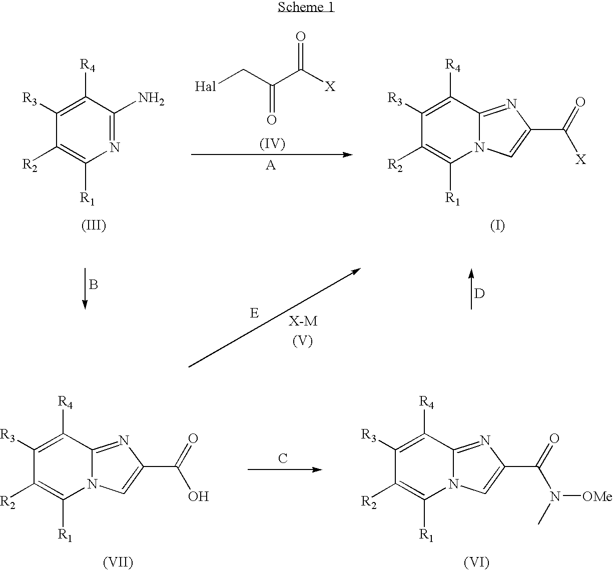

Route A consists in preparing the 2-aminopyridines of formula (III) according to the methods known to a person skilled in the art and in forming the imidazo[1,2-a]pyridine ring by condensation with a 1-arylpropane-1,2-dione derivative (IV) in which Hal represents a halogen, for example according to the method described by J-J. Bourguignon et al. in Aust. J. Chem. 1997, 50, 719-725.

The second synthesis route B, C, D consists in reacting an organometallic derivative of general formula (V) in which X is defined as above and M represents a lithium atom or a Mg-Hal group with a Weinreb amide of formula (VI), the reactive functional groups of which are optionally protected, according to methods known to a person skilled in the art as described in Nahm, S.; Weinreb, S. M., Tetrahedron Letters (1981), 22(39), 3815-18 and in Sibi, M. P. Organic Preparations and Procedures Int. 1993, 25, 15-40. The Weinreb amide of formula (VI) is obtained by coupling the acid derivative of formula (V) or one of its reactive derivatives with an N,O-dialkylamine according to the methods described in the references above.

The coupling may be carried out in the presence of a coupling agent such as CDI, EDCI, HATU or HBTU and a base such as diisopropylethylamine, triethylamine or pyridine, in an inert solvent such as THF, DMF or dichloromethane. Alternatively, it is possible to react the N,O-dialkylamine with an ester of the acid of formula (V) in the presence of a catalyst such as trimethylaluminum (Weinreb. S. M. Synth. Commun. 1982, 12, 989).

It is also possible, according to a third synthesis route (B, E), to react the organometallic derivative of general formula (V) defined as above with an imidazo[1,2-a]pyridine-2-carboxylic acid of general formula (VII) in which R₁, R₂, R₃ and R₄ are defined as before or one of the reactive derivatives or salts thereof such as ester, acid halide, anhydride or amide according to methods known to a person skilled in the art, as described in J. March, Advanced Organic Chemistry (Wiley, 5th Ed. 2001) p. 567 and 1213 or in the references cited.

The products of formula (I) may be subjected, if desired and if necessary, to obtain products of formula (I) or to be converted to other products of formula (I), to one or more of the following conversion reactions, in any order:
a) a reaction for converting a hydroxyl functional group to an alkoxy functional group;
b) a reaction for the catalytic coupling of a halogenated derivative and of an organometallic derivative such as a stannic or boronic derivative, in order to introduce a methyl substituent;
c) a reaction for protecting reactive functional groups;
d) a reaction for removing the protecting groups that may be carried by the protected reactive functional groups;
e) a reaction for forming a salt with a mineral or organic acid or with a base to obtain the corresponding salt.

In scheme 1, the starting compounds and the reactants, when their preparation method is not described, are commercially available or are described in the literature, or else may be prepared according to methods that are described therein or that are known to a person skilled in the art.

### EXAMPLES

The following examples describe the preparation of certain compounds according to the invention. These examples are not limiting and serve only to illustrate the present invention. The numbers of the examples refer to those given in the tables below, which illustrate the chemical structures and the spectroscopic characteristics of some of the compounds according to the invention.

### Example 1

(5-Methoxyimidazo[1,2-a]Pyridin-2-yl)-(Phenyl)Methanone Added to a solution of 110 mg of (5-bromoimidazo[1,2-a]pyridin-2-yl) (phenyl)methanone in 14 ml of methanol were 268 mg of sodium methylate and 108 mg of copper powder. The mixture was heated for 45 minutes at 120° C. in a microwave then cooled and treated with 20 ml of water and concentrated to dryness. The residue was taken up in dichloromethane. The insoluble material was removed and the filtrate concentrated to dryness. The residue was chromatographed on a silica column while eluting with a 97/3 mixture of dichloromethane and methanol. The fractions containing the expected product were combined and concentrated to dryness under reduced pressure to give 26 mg of (5-methoxyimidazo[1,2-a]-pyridin-2-yl) (phenyl)methanone in the form of a yellow solid.

### Example 2

(7-Methoxyimidazo[1,2-a]Pyridin-2-yl)-(Phenyl)Methanone

Added to a solution of 110 mg of (7-hydroxyimidazo[1,2-a]pyridin-2-yl) (phenyl)methanone hydrobromide (1:1) in 10 ml of acetone were 96 mg of potassium carbonate and 78 mg of methyl iodide. The reaction mixture was heated under reflux for 15 hours then concentrated to dryness. After chromatography on a silica column while eluting with a 96/4 mixture of dichloromethane and methanol, the fractions containing the expected product were combined and concentrated to give 44 mg of (7-methoxy-imidazo[1,2-a]pyridin-2-yl)(phenyl)methanone in the form of a pale yellow solid.

### Example 3

(6-Chloroimidazo[1,2-a]Pyridin-2-yl)-(Phenyl)Methanone Hydrobromide (1:1)

Added dropwise to a solution of 0.82 g of 3-bromo-1-phenylpropane-1,2-dione in 3 ml of DMF cooled to 4° C. was a solution of 386 mg of 2-amino-5-chloropyridine in 7 ml of DMF. The reaction mixture was stirred for 6 hours at 4° C., then kept at the same temperature without stirring for 64 hours at 4° C. The precipitate was filtered and washed with diethyl ether then suspended in 10 ml of ethanol. The reaction mixture was heated under reflux for 2 hours then concentrated under reduced pressure. The residue was taken up in diethyl ether and triturated then filtered and washed with diethyl ether. 0.235 g of (6-chloroimidazo[1,2-a]pyridin-2-yl) (phenyl)methanone hydrobromide (1:1) was obtained in the form of a beige solid.

The intermediates described below are useful for preparing the compounds of the present invention.

(7-Hydroxyimidazo[1,2-a]Pyridin-2-yl)(Phenyl)Methanone Hydrobromide (1:1)

Added to a suspension of 0.250 g of 4-hydroxypyridine-2-amine in 4 ml of THF was a solution of 0.619 g of 3-bromo-1-phenylpropane-1,2-dione in 4 ml of THF. The reaction mixture was stirred for 15 h at 20° C., then heated under reflux for 3 hours and concentrated to dryness. The residue was taken up in methanol and filtered over a cation exchange cartridge (Bond Elut SCX Varian, 5 g). The fractions containing the expected product were combined and concentrated. The product was purified by chromatography on a silica column while eluting with a 95/5 mixture of dichloromethane and methanol. The fractions containing the expected product were combined and concentrated to give 55 mg of (7-hydroxyimidazo[1,2-a]pyridin-2-yl)(phenyl)methanone hydrobromide (1:1) in the form of a beige solid.

¹H NMR spectrum (DMSO-d6, δin ppm): 6.67 (dd, J =2.5 and 7.5 Hz, 1H); 6.75 (d, J=2.5 Hz, 1H); 7.55 (broad t, J=7.5 Hz, 2H); 7.65 (broad t, J=7.5 Hz, 1H); 8.26 (broad d, J=8.0 Hz, 2H); 8.40 (s, 1H); 8.43 (d, J=7.5 Hz, 1H); 10.5 (s, 1H)

Mass spectrum (EI): m/z 238 (base peak): [M^{+.}], m/z 210: [M^{+.}]-[CO], m/z 105: PhCO⁺.

IR spectrum (KBr) : 3165; 2597; 1637; 1551; 1234; 1160; 907; 714 & 698 cm⁻¹.

5-Bromoimidazo[1,2-a]Pyridin-2-yl)(Phenyl)Methanone

5-Bromoimidazo[1,2-a]pyridin-2-yl)(phenyl)methanone is obtained in a similar manner by replacing the 4-hydroxypyridine-2-amine with 2-amino-6-bromopyridine.

¹H NMR spectrum (DMSO-d6, δin ppm): 7.39 (dd, J=7.5 and 9.0 Hz, 1H); 7.47 (broad d, J=7.5 Hz, 1H); 7.59 (broad t, J=7.5 Hz, 2H); 7.70 (broad t, J=7.5 Hz, 1H); 7.82 (broad d, J=9.0 Hz, 1H); 8.33 (broad d, J =8.0 Hz, 2H); 8.48 (s, 1H)

Mass spectrum (LCMS): m/z 300 (base peak): [M+H]+.

IR spectrum (KBr): 3156; 1639; 1511; 1260; 1237; 1179; 1125; 895; 775; 705 & 697 cm⁻¹.

### 4,5-Dimethoxypyridine-2-Amine

Added to a solution of 0.316 g of sodium carbonate in 8 ml of water were 0.48 g of 4,5-dimethoxy-2-pyridine-methanol then 0.529 g of potassium permanganate was added in portions so as to keep the temperature below 22° C. After stirring for 2 hours at 20° C., the reaction medium was filtered and the insoluble material was rinsed with water. The filtrate was brought to a pH below 1 by addition of 5N hydrochloric acid then concentrated to dryness under reduced pressure. The residue was taken up in 16 ml of tert-butanol. After addition of 0.734 ml of diphenylphosphoryl azide and 0.95 ml of triethylamine, the reaction medium was heated at 80° C. for 16 hours then brought to 20° C. The insoluble material was filtered and the filtrate concentrated to dryness under reduced pressure. The residue was triturated with methanol, the insoluble material was removed and the filtrate concentrated to dryness. The residue was taken up in 10 ml of dichloromethane and treated with 2 ml of trifluoroacetic acid for 16 hours at 20° C. After evaporation, the residue was purified by filtration over a cation exchange cartridge (Bond Elut SCX Varian, 2 g) while eluting with 3.5N ammoniacal methanol. The fraction containing the expected product was concentrated and the residue chromatographed over a silica cartridge while eluting with a 95/5 mixture of dichloromethane and methanol. The fractions containing the expected product were concentrated under reduced pressure to give 0.147 g of 4,5-dimethoxypyridine-2-amine in the form of a beige solid.

¹H NMR spectrum (DMSO-d6, δin ppm): 3.65 (s, 3H); 3.72 (s, 3H); 5.42 (broad s, 2H); 6.07 (s, 1H); 7.48 (s, 1H).

Mass spectrum (ES): m/z=155 [MH]+(base peak)

The following tables illustrate the chemical structures (table 1) and the spectroscopic characteristics (table 2) of some of the compounds according to the invention. The tables repeat the compound numbers from the above examples.

TABLE 1

(I)

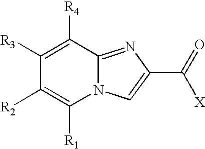

| Compound | R₁ | R₂ | R₃ | R₄ | X | Salt |
|---|---|---|---|---|---|---|
| 1 | OMe | H | H | H | Ph | |
| 2 | H | H | OMe | H | Ph | |
| 3 | H | Cl | H | H | Ph | HBr |
| 4 | H | OMe | OMe | H | Ph | HBr |
| 5 | H | OMe | H | H | Ph | |
| 6 | H | H | Me | H | Ph | |
| 7 | H | H | H | H | H | HCl |

TABLE 2

| Compound | Characterization |
|---|---|
| 1 | ¹H NMR spectrum (DMSO-d6, δ in ppm): 4.14 (s, |
| | 3H); 6.49 (d, J = 7.5 Hz, 1H); 7.35 (d, J = 9.0 Hz, |
| | 1H); 7.45 (dd, J = 7.5 and 9.0 Hz, 1H); |
| | 7.58 (t, J = 7.5 Hz, 2H); 7.68 (t, J = 7.5 Hz, |
| | 1H); 8.31 (m, 2H). |
| | Mass spectrum (EI): m/z 252 (base peak): [M^{+.}], |
| | m/z 237: [M^{+.}] − CH₃, m/z 209: 237 − [CO], m/z 105: |
| | PhCO⁺, m/z 77: Ph⁺ |
| | IR spectrum (KBr): 3172; 2946; 1643; 1545; |
| | 1529; 1270; 1234; 1106; 975; 899; 771; 731 & |
| | 713 cm⁻¹. |
| 2 | ¹H NMR spectrum (DMSO-d6, δ in ppm): 3.86 (s, |
| | 3H); 6.76 (dd, J = 2.5 and 7.5 Hz, 1H); |
| | 7.05 (d, J = 2.5 Hz, 1H); 7.56 (broad t, J = 7.5 Hz, |
| | 2H); 7.66 (broad t, J = 7.5 Hz, 1H); |
| | 8.30 (broad d, J = 8.0 Hz, 2H); 8.47 (m, 2H) |
| | Mass spectrum (EI): m/z 252 (base peak): [M^{+.}], |
| | m/z 224: [M^{+.}] − [CO], m/z 237: [M^{+.}] − CH₃, m/z |
| | 209: 237⁺ − [CO], m/z 105: PhCO⁺, m/z 77: Ph⁺. |
| | IR spectrum (KBr): 3159; 1653; 1548; 1491; |
| | 1335; 1236; 1212; 1173; 1018; 897; 714 & 681 cm⁻¹. |
| 3 | ¹H NMR spectrum (CDCl₃-d1, δ in ppm): from 7.51 |
| | to 7.64 (m, 3H); 7.71 (broad t, J = 7.5 Hz, |
| | 1H); 7.81 (d, J = 9.5 Hz, 1H); 8.27 (broad d, |
| | J = 8.0 Hz, 2H); 8.64 (s, 1H); 8.92 (d, J = 2.0 Hz, |
| | 1H). |
| | Mass spectrum (EI): m/z 256: [M^{+.}], m/z 228: |
| | [M^{+.}] − [CO]. |
| | IR spectrum (KBr): 3067; 2792; 1655; 1546; |
| | 1438; 1289; 1268; 1244; 1088; 916; 811 & 725 cm⁻¹ |
| 4 | ¹H NMR spectrum (DMSO-d6, δ in ppm): 3.88 (s, |
| | 3H); 4.03 (s, 3H); 7.08 (s, 1H); 7.65 (broad t, |
| | J = 7.5 Hz, 2H); 7.77 (broad t, J = 7.5 Hz, |
| | 1H); 8.10 (broad d, J = 8.0 Hz, 2H); 7.44 (s, |
| | 1H); 8.62 (s, 1H). |
| | IR spectrum (KBr): 3284, 1660, 1597, 1563, |
| | 1447, 1439, 1316, 1285, 1266, 1239, 1227, 992 cm⁻¹ |
| | Mass spectrum (EI): m/z = 282 [M]⁺ (base peak), |
| | m/z = 267 [M − CH₃]⁺, m/z = 239 [m/z = 267 − CO]⁺, |
| | m/z = 105 [C₇H₅O]⁺, m/z = 77 [C₆H₅]⁺. |

The compounds according to the invention were the subject of pharmacological assays for determining their modulatory effect on NOT.

Evaluation of the in vitro Activity on N2A Cells

Assays consisted in measuring the in vitro activity of the compounds of the invention on a cell line (N2A) endogenously expressing the mouse Nurrl receptor and stably transfected with the NOT binding response element (NBRE) coupled to the luciferase reporter gene. The EC₅₀ values are between 0.01 and 1000 nM. The assays were carried out according to the procedure described below.

The Neuro-2A cell line comes from a standard commercial source (ATCC) . The Neuro-2A clone was obtained from a spontaneous tumor originating from an A albino mouse strain, by R. J Klebe et al. This Neuro-2A line is subsequently stably transfected with 8NBRE-luciferase. The N2A-8NBRE cells are cultured until confluence in 75 cm² culture flasks containing DMEM supplemented with 10% of fetal calf serum, 4.5 g/l of glucose and 0.4 mg/ml of geneticin. After a week of culture, the cells are recovered with 0.25% trypsin for 30 seconds and then resuspended in DMEM without phenol red, containing 4.5 g/l of glucose and 10% of Hyclone delipidized serum, and deposited into transparent-bottom 96-well white plates. The cells are deposited at a rate of 60 000 per well in 75 μl for 24 hours before the addition of the products. The products are applied in 25 μl and incubated for a further 24 hours. On the day of the measurement, an equivalent volume (100 μl ) of Steadylite is added to each well and then left for a period of 30 minutes in order to obtain complete cell lysis and maximum signal production. The plates are subsequently measured in a luminescence counter for microplates after having been sealed with an adhesive film. The products are prepared in the form of a stock solution at 102 M and then diluted in 100% of DMSO. Each product concentration is prediluted in culture medium before incubation with the cells, thus containing 0.625% final concentration of DMSO.

For example, compounds No. 7 and 6 showed an EC₅₀ value of 31 nM and 1.2 nM, respectively.

### Evaluation of the Binding to the Human NOT Receptor

The direct binding between compounds of the invention and the human NOT receptor was evaluated using SPR (surface plasmon resonance) technology. In this assay, the protein is immobilized covalently to the matrix and the molecule to be studied is injected into the chamber containing the sensor chip. The signal is directly proportional to the amount of product bound to the protein. The binding assays were carried out in a BIACORE S51 instrument (Biacore Inc., Piscataway N.J.). The GST-NOT (NOT-FL) whole protein was provided by Invitrogen (PV3265). The NOT ligand-binding domain (His-Thr-NOT 329-598) was expressed and purified as described in Nature 423, 555-560. The two proteins, diluted to a concentration of 20 μg/ml in an acetate buffer, pH 5.0, containing 5 mM of DTT, were immobilized on a carboxymethyl 5′ dextran surface (CM5 sensor chip, Biacore Inc.) by amine coupling according to the protocol recommended by Biacore, elution being carried with an HBS-N buffer (10 mM HEPES, 0.15 M NaCl, 3 mM EDTA, pH 7.4). Approximately 10000-15000 resonance units (RU) of the proteins are captured on the surface of the CM5 sensor chip. The stock solutions of the compounds to be studied, at 1.5 mM in DMSO, are serially diluted in elution buffer (50 mM HEPES pH 8; 150 mM NaCl; 10 MM MgCl₂; 2% DMSO, 1 mM DTT) to concentrations ranging from 3.75 to 0.1 μM. Each product concentration is injected at 4° C. for 1 minute at 30 μl/min. The dissociation was recorded for 5 minutes without any other procedure for regenerating the surface. The signals obtained are corrected by testing each product concentration on an unmodified dextran surface (blank). The signal due to the migration buffer is deducted from the total signal (“double referencing”) as is the effect of the DMSO. The signal analysis is carried out using the Biacore S51 analytical software (version 1.2.1). The compounds are subsequently classified according to their maximum binding level and to kinetic parameters for binding to the immobilized protein.

By way of example, compound No. 6 has a medium affinity and compound No. 3 has a high affinity.

It therefore appears that the compounds according to the invention have a NOT-modulating effect.

The compounds according to the invention can therefore be used for the preparation of medications for their therapeutic use in the treatment or prevention of diseases involving NOT receptors.

These medications find their use in therapeutics, in particular in the treatment and prevention of neurodegenerative diseases, such as, for example, Parkinson's disease, Alzheimer's disease, tauopathies (for example, progressive supranuclear palsy, frontotemporal dementia, corticobasal degeneration, Pick's disease), multiple sclerosis; cerebral traumas such as ischemia and cranial traumas and epilepsy; psychiatric diseases such as schizophrenia, depression, substance dependency, attention deficit hyperactivity disorders; inflammatory diseases such as vascular pathologies, atherosclerosis, inflammations of the joints, arthrosis, rheumatoid arthritis, osteoarthritis, allergic inflammatory diseases such as asthma, and to conclude the treatment of osteoporosis and cancers.

These compounds could also be used as a treatment associated with stem cell transplants and/or grafts.

According to another of its aspects, the present invention relates to pharmaceutical compositions comprising, as active ingredient, a compound according to the invention. These pharmaceutical compositions contain an effective dose of at least one compound according to the invention, or a pharmaceutically acceptable salt of said compound, and also at least one pharmaceutically acceptable excipient.

Said excipients are selected according to the pharmaceutical form and the method of administration desired, from the usual excipients which are known to those skilled in the art.

In the pharmaceutical compositions of the present invention for oral, sublingual, subcutaneous, intramuscular, intravenous, topical, local, intratracheal, intranasal, transdermal or rectal administration, the active ingredient of formula (I) above, or its salt, can be administered in unit administration form, as a mixture with conventional pharmaceutical excipients, to animals or to human beings for the prophylaxis or the treatment of the disorders or the diseases above.

The appropriate unit administration forms include oral forms such as tablets, soft or hard gel capsules, powders, granules and oral solutions or suspensions, sublingual, buccal, intratracheal, intraocular and intranasal administration forms, forms for administration by inhalation, topical, transdermal, subcutaneous, intramuscular or intravenous administration forms, rectal administration forms, and implants. For topical application, the compounds according to the invention can be used in creams, gels, ointments or lotions.

By way of example, a unit administration form of a compound according to the invention in tablet form can comprise the following components:

| | | |
|---|---|---|
| Compound according to the invention | 50.0 | mg |
| Mannitol | 223.75 | mg |
| Sodium croscarmellose | 6.0 | mg |
| Maize starch | 15.0 | mg |
| Hydroxypropyl methyl cellulose | 2.25 | mg |
| Magnesium stearate | 3.0 | mg |

There may be specific cases where higher or lower dosages are appropriate; such dosages do not depart from the scope of the invention. According to customary practice, the dosage appropriate for each patient is determined by the physician according to the method of administration and the weight and the response of said patient.

According to another of its aspects, the present invention also relates to a method for treating the pathologies indicated above, which comprises the administration, to a patient, of an effective dose of a compound according to the invention, or one of its pharmaceutically acceptable salts.

We claim:

1. A method for treating or preventing a disease in which the NOT receptor is involved, a neurodegenerative disease, multiple sclerosis, cerebral trauma, epilepsy, a psychiatric disease, an inflammatory disease, osteoporosis, cancer, Parkinson's disease, Alzheimer's disease, tauopathy, schizophrenia, depression, substance dependency or attention deficit hyperactivity disorder, in a patient in need thereof, comprising administering to the patient a pharmaceutically effective amount of a compound of formula (I): 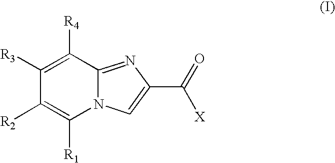
wherein:
X is phenyl, and R₁, R₂, R₃ and R₄ are hydrogen; or
X is phenyl, R₃ is methyl, and R₁, R₂ and R₄ are hydrogen; or
X is phenyl, R₂ is chlorine or methoxy, and R₁, R₃ and R₄ are hydrogen; or
X is phenyl, R₂ and R₃ are methoxy, and R₁ and R₄ are hydrogen; or
X is phenyl, R₁ is methoxy, and R₂, R₃ and R₄ are hydrogen; or
X is phenyl, R₃ is methoxy, and R₁, R₂ and R₄ are hydrogen; or
X is 4-methylphenyl, R₂ is methyl, and R₁, R₃ and R₄ are hydrogen; or
X is 4-chlorophenyl, R₁ is chlorine, methoxy or methyl, and R₂, R₃ and R₄ are hydrogen; or
X is 4-chlorophenyl, R₂ is chlorine or methyl, and R₁, R₃ and R₄ are hydrogen; or
X is 4-chlorophenyl, R₃ is methyl, and R₁, R₂ and R₃ are hydrogen; or
X is 4-chlorophenyl, R₄ is methyl, and R₁, R₂ and R₄ are hydrogen; or
X is 4-chlorophenyl, R₁ and R₃ are methyl, and R₂ and R₄ are hydrogen; or
X is 4-chlorophenyl, and R₁, R₂, R₃ and R₄ are hydrogen; or
X is 2-chlorophenyl, and R₁, R₂, R₃ and R₄ are hydrogen; or
X is 4-methylphenyl, and R₁, R₂, R₃ and R₄ are hydrogen, or an addition salt with a pharmaceutically acceptable acid thereof.

2. The method according to claim 1, wherein for the compound of formula (I):
X is phenyl, and R₁, R₂, R₃ and R₄ are hydrogen; or
X is phenyl, R₂ is chlorine or methoxy, and R₁, R₃ and R₄ are hydrogen.

3. The method according to claim 1, for treating or preventing a neurodegenerative disease.

4. The method according to claim 2, for treating or preventing a neurodegenerative disease.

5. The method according to claim 1, for treating or preventing multiple sclerosis, cerebral trauma or epilepsy.

6. The method according to claim 2, for treating or preventing multiple sclerosis, cerebral trauma or epilepsy.

7. The method according to claim 1, for treating or preventing a psychiatric disease.

8. The method according to claim 2, for treating or preventing a psychiatric disease.

9. The method according to claim 1, for treating or preventing an inflammatory disease.

10. The method according to claim 2, for treating or preventing an inflammatory disease.

11. The method according to claim 1, for treating or preventing osteoporosis or cancer.

12. The method according to claim 2, for treating or preventing osteoporosis or cancer.

13. The method according to claim 1, for treating or preventing Parkinson's disease, Alzheimer's disease or tauopathy.

14. The method according to claim 2, for treating or preventing Parkinson's disease, Alzheimer's disease or tauopathy.

15. The method according to claim 1, for treating or preventing schizophrenia, depression, substance dependency, or attention deficit hyperactivity disorder.

16. The method according to claim 2, for treating or preventing schizophrenia, depression, substance dependency, or attention deficit hyperactivity disorder.
